(19) United States
(12) Patent Application Publication
(10) Pub. No.: US 2005/0043692 A1
(43) Pub. Date: Feb. 24, 2005

(54) Topical eye drop holder (TEDH)

(76) Inventor: Wong Seng Ling, Sitiawan (MY)

Correspondence Address: Ling Wong Seng, 136, Taman Raja Shariman, Sitiawan, Perak Darul Ridzuan, 32000, MY

(21) Appl. No.: 10/914,301

(22) Filed: Aug. 10, 2004

(30) Foreign Application Priority Data
Dec. 2, 2002 (MY) 20024508

(51) Int. Cl. A61M 35/00

(52) U.S. Cl. 604/289

(57) ABSTRACT

The TEDH comes in a single complete unit. There is no movable or detachable part. The device is very safe to use by people of all ages. The raised marking ↑ (up) helps the visually impaired person to fix the position over the eye when the device is used. An observer can see the drop of medication dripping down from the tip of the dropper through the transparent plastic holder. TEDH is designed to help the patient to apply the medication into the lower eye-lid and not on the eyeball.

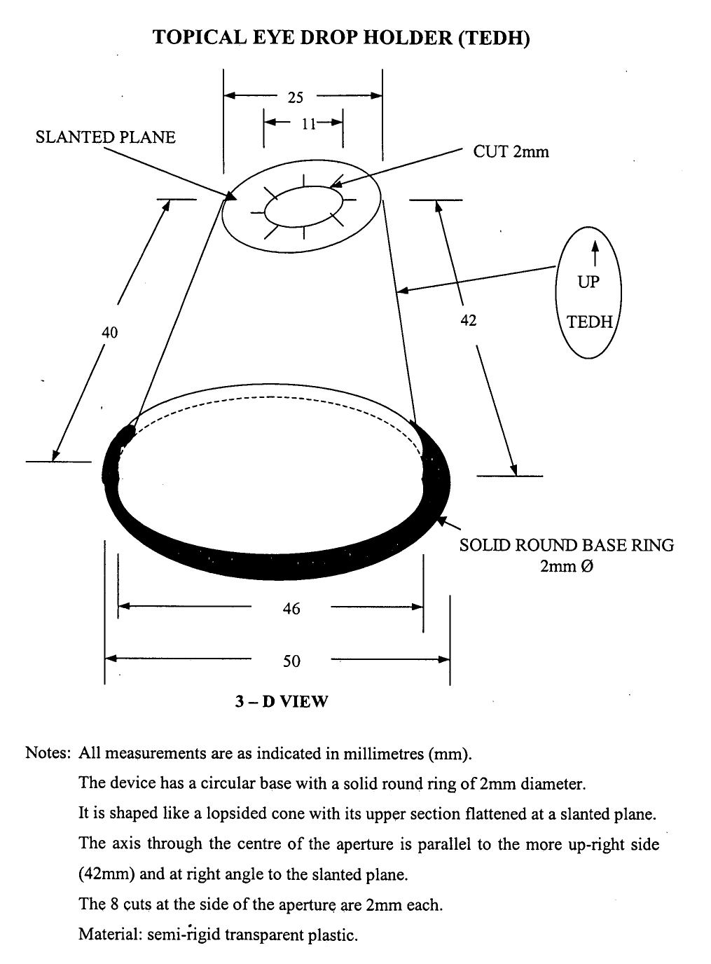

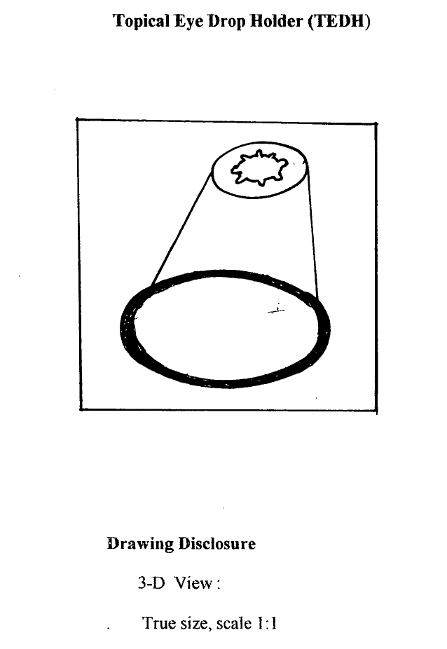

### TECHNICAL FIELD

This invention relates to a device specifically designed for dripping liquid eye medication from an eye drop bottle into the eye safely.

### BACKGROUND ART

For many people who suffer from eye related problems and diseases, applying the liquid eye medication to the eye regularly, several times a day by themselves, is a difficult task. Old and elderly people are particularly affected when they cannot control their hands from shaking. They hold the dropper bottle over the open eye tying to drip a drop of the medication into the eye and in the process, the tip of the dropper touches the eye causing trauma and injury to the cornea of the eye. The injury may cause blindness eventually.

There is no known simple device yet to help the patients hold the dropper securely over the eye.

### DISCLOSURE OF INVENTION

The present invention aims to provide a device for holding the eye medication dropper firmly over the eye when applying the liquid eye medication into the lower eyelid without causing any physical harm to any part of the eye.

The aperture of 11 mm diameter and 8 regular cuts of 2 mm each at its side at the top slanted plane of the device will be able to accommodate most eye medication droppers being used today.

By firmly pushing the head of the medication dropper into the aperture, the dropper is held firmly in the device. When the device with the dropper attached is placed over the eye, there is sufficient clearance between the tip of the dropper and the eyeball.

When the device is used as directed, the drop of medication dripping from the dropper can be clearly observed. In this manner, overdose of medication can be avoided.

The dropper cap can be screwed on to the dropper without the need to remove and detach the dropper from the device. In this manner, contamination to the medication in the dropper bottle and the dropper tip is prevented.

When the dropper cap is screwed on the dropper in the device, it forms a unit item that can be stored easily, preferably in a refrigerator to preserve the medication.

The solid round band at the base of the device helps to push open the lower eye-lid when the device is placed over the eye and a slight downward pressure is applied to it.

The material suitable for making the device is semi-rigid transparent plastic. The device may be manufactured through an injection process by using a mould.

### DESCRIPTION OF DRAWINGS and BEST MODE OF CARRYING OUT THE INVENTION

In order that the invention may be more readily understood and put into practical effect, a preferred example of the invention will now be described with reference to the accompanying drawing on page 6.

The drawing illustrates the view of the device in 3-D dimensions with its accompanying notes.

All measurements are indicated in millimeters (mm).

Material: semi-rigid transparent plastic commonly known in the industry as P.P.

Manufacture: By injection mould process, subject to Engineer's details.

1. The Topical Eye Drop Holder (TEDH) is specifically designed for instilling liquid eye medication into the eye safely.

2. Any eye medication dropper can be placed in the TEDH to instill the medication into the eye.

3. The solid round band at the base of the TEDH helps to push open the lower eyelid for the eye drop to drip into it, without having to push the eyelid down with a finger.

4. The TEDH protects the eye, particularly the cornea from any physical injury when applying the eye drops

5. The transparent plastic allows observation of the drop of medication dripping down from the tip of the dropper into the eye to prevent any overdose and wastage.

6. Old and elderly people who suffer from eye-related diseases and problems will need to apply the eye medication regularly, several times a day. They can use the TEDH to apply the eye drops by themselves confidently without the need of assistance from another person. They will be able to maintain their independence and retain their self-confidence and self-esteem.
